(19) United States
(12) Patent Application Publication
(10) Pub. No.: US 2017/0239055 A1
(43) Pub. Date: Aug. 24, 2017

(54) ENTIRELY CERAMIC ACETABULUM HAVING A POROUS REAR OUTER SURFACE

(71) Applicant: CeramTec GmbH, Plochingen (DE)

(72) Inventors: Roman PREUSS, Kirchheim unter Teck (DE); Marita RASCHKE, Leonberg (DE); Peter STRÖTGEN, Königsfeld (DE); Tobias WEISS, Ebersbach (DE)

(21) Appl. No.: 15/324,480

(22) PCT Filed: Jul. 1, 2015
(86) PCT No.: PCT/EP2015/064928

§ 371 Date: May 10, 2017

(30) Foreign Application Priority Data
Jul. 9, 2014 (DE) 102014213362.9

(51) Int. Cl. A61F 2/34; A61L 27/30; A61L 27/34; A61L 27/32; A61L 27/56; A61L 27/10

(52) U.S. Cl. CPC A61F 2/34; A61L 27/56; A61L 27/10; A61L 27/34; A61L 27/32; A61L 27/306; A61F 2002/30968

(57) ABSTRACT

The invention relates to an acetabulum (1) for a hip prosthesis, comprising an inner cavity (2) and a rear outer surface (3) facing the bone. In order to be able to implant the acetabulum (1) without using cement, at least some areas of the outer surface (3) are porous and osseointegrative, and the acetabulum (1) is made exclusively of a ceramic material.

The invention relates to an acetabulum for a hip joint prosthesis having an inner cavity and a rear outer surface facing the bone.

The current clinical prior art provides for ceramic sliding surfaces for endoprosthetic acetabula only in combination with metallic components (modular systems). The metallic components (acetabulum) are frequently provided with a likewise metallic osseointegrative coating, which allows the direct ingrowth of the bone into the implant surface. A ceramic component (insert) on the other hand ensures low wear of the sliding pair.

Disadvantages of the modular systems include the relatively large wall thickness of the complete system with respect to the bone resection, as well as application errors by tilted insertion of the ceramic seats in the metal socket, which can lead to ceramic fracture.

A reduction in the wall density of the modular systems is limited due to deformation of the acetabulum that occurs during impacts, as a result of which the insertion of the ceramic component can become difficult or completely impossible.

Apart from use of the above modular hip joint endoprostheses on the acetabular side with ceramic sliding surfaces, the market also includes various directly implantable metallic monolithic acetabular solutions (including osseointegrative coating) with a metallic sliding surface, with the goal of the bone-sparing operation.

The use of a directly implantable full-ceramic acetabulum with integrated porous osseointegrative rear outer surface constitutes an advantageous novelty.

Such a metal-free acetabulum combines the advantages of the bone-sparing operation with those of a biocompatible material, low wear of the sliding pair, and excellent osseointegration.

An acetabulum according to the invention for a hip joint prosthesis with an inner cavity and a rear outer cavity facing the bone is characterized in that the outer surface is designed to be porous and osseointegrative at least partially, and the acetabulum consists solely of a ceramic material.

A method according to the invention for producing the porous osseointegrative outer surface of the acetabulum is characterized in that the acetabulum is produced from a sintered molded article and that a porous osseointegrative layer is applied to the outer surface at last in some regions.

In an embodiment according to the invention, the porous outer surface is created by application of a ceramic slurry and place-holders (pore-formers) in the green state and burning off of the place-holders during sintering. Preferably the porous layer can be created by foaming of a slurry in the green state and subsequent sintering. By means of the sintering process, a bond is achieved with the outer surface, and an open-pored structure is created with roughened surface, which advantageously supports the accretion and ingrowth of the bone. This type of coating in combination with the ceramic acetabulum is likewise metal-free and is thereby advantageous with respect to allergic reactions.

The ceramic slurry consists of the same material as the acetabulum. The complete acetabulum including coating is thereby completely free of metals and is advantageous with respect to allergic reactions.

The porous outer surface can also be created by joining of a dense molded article with a porous molded article in sintered state. The porous molded article can be created by foaming of ceramic slurries and sinters or infiltration of a porous substrate material with slurry and sinters. The joining is preferably carried out by soldering or gluing.

The porous osseointegrative outer surface can be produced according to the invention also by 2c-injection molding (dense phase and porous or pore-forming phase) and sintering.

The porous outer surface through further coating can preferably acquire additional functionality, such as higher integration speed or antibacterial effects. Integration speed is understood to mean the time required for ingrowth with the bone. Preferably this can be achieved with bioglasses, hydroxylapatite, functionalized proteins, or hydrogels.

The acetabulum according to the invention consists entirely of a ceramic material, and can be directly implanted due to the porous outer surface. The bone grows quickly into the porous outer surface, so that a firm seat is ensured quickly after the operation. No bone cement is used in the implantation.

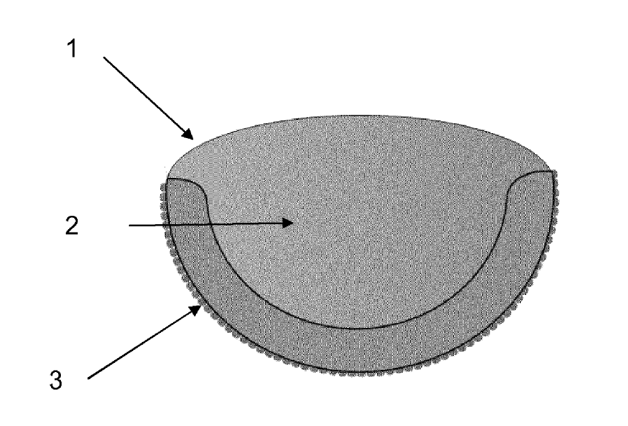

FIG. 1 shows an acetabulum 1 with an inner cavity 2 and a rear outer surface 3 facing the bone. According to the invention the outer surface 3 is designed to be porous and osseointegrative at least in places. The acetabulum 1 consists solely of a ceramic or a ceramic material.

1. An acetabulum for a hip joint prosthesis with an inner cavity and a rear outer surface facing the bone, wherein the outer surface is designed to be porous and osseointegrative at least in places, and the acetabulum consists solely of a ceramic material.

2. A method for producing the porous osseointegrative outer surface of the acetabulum according to claim 1, wherein the acetabulum is produced from a sintered molded article and the porous osseointegrative layer is applied to the outer surface at least in places.

3. The method according to claim 2, wherein the porous outer surface is produced by application of a ceramic slurry and place-holders (pore-formers) in the green state and burning off the place-holders during sintering.

4. The method according to claim 2, wherein the porous outer surface is produced by foaming of a ceramic slurry in the green state and subsequent sintering.

5. The method according to claim 2, wherein the porous outer layer is produced by joining of a dense molded article to a porous ceramic molded body in the sintered state.

6. The method according to claim 5, wherein the porous molded body is produced by foaming of ceramic slurries and sinters or infiltration of a porous substrate material with slurry and sinters.

7. The method according to claim 5, wherein the joining is carried out by soldering or gluing.

8. The method according to claim 2, wherein the porous outer surface is produced by 2c-injection molding (dense phase and porous or pore-forming phase) and sintering.

9. The method according to claim 2, wherein the porous outer surface acquires additional functionality through further coating, such as higher integration speed or antibacterial effects.

10. The method according to claim 9, wherein the porous rear face is coated with bioglasses, hydroxylapatite, functionalized proteins, or hydrogels.
